(19) United States
(12) Patent Application Publication
(10) Pub. No.: US 2007/0072903 A1
(43) Pub. Date: Mar. 29, 2007

(54) Altering the concentration of intracellular agents by p2x7 receptor modulation

(76) Inventors: James Elliott, London (GB); Christopher Higgins, London (GB)

Correspondence Address: MORGAN LEWIS & BOCKIUS LLP, 1111 PENNSYLVANIA AVENUE NW, WASHINGTON, DC, 20004, US

(21) Appl. No.: 10/574,196

(22) PCT Filed: Oct. 4, 2004
(86) PCT No.: PCT/GB04/04215

§ 371 Date: Oct. 3, 2006

(30) Foreign Application Priority Data
Oct. 3, 2003 (GB) 0323147.9

(51) Int. Cl. A61K 31/47

(52) U.S. Cl. 514/310

(57) ABSTRACT

Disclosed is an in vitro method of increasing the effective intracellular concentration of a therapeutic molecule within a cell expressing a P2X₇ receptor, the method comprising the step of contacting the cell with the therapeutic molecule and with a substance which stimulates the P2X₇ receptor.

### FIELD OF THE INVENTION

This invention relates to a method of inhibiting efflux proteins, pharmaceutical compositions for performing the method, and the use of certain active agents in the preparation of medicaments to inhibit efflux proteins.

### BACKGROUND OF THE INVENTION

Plasma membrane lipids are distributed asymmetrically in normal cells, with anionic phospholipids such as phosphatidylserine (PS) usually being confined to the inner leaflet of the plasma membrane. Energy is expended to maintain this asymmetry against a background of passive spontaneous, albeit slow, phospholipid diffusion between leaflets, and though no protein has uniquely been identified with the function of the inward transport of PS (aminophospholipid translocase activity), candidate proteins have been identified (Williamson and Schlegel, Biochim. Biophys. Acta 2002, 53-63).

Loss of membrane asymmetry with the consequent exposure of PS at the cell surface is known to occur (i) on anucleate ‘cells’ such as platelets and to a lesser extent erythrocytes as an integral part of the clotting cascade; and (ii) prior to membrane breakdown, being a marker of apoptotic cell death (Martin et al, 1995 J. Exp. Med. 182, 1545-1556; Vermes et al, 1995 J. Immunol. Methods 184, 39-51). Indeed binding of fluorescent annexin V to PS is the basis of commonly used commercially available assays for the detection of apoptotic cells. Reported exceptions to rule that PS exposure indicates apoptotic are rare, though it has been found to occur during bicarbonate-stimulated ‘capacitation’ of sperm required for fertilisation (Gadella & Harrison, 2002 Biol. Reprod. 67, 340-350), whilst PS redistribution on macrophages is also believed necessary for engulfment of apoptotic cells (Marguet et al, 1999 Nat. Cell Biol. 1, 454-456). Other than in such exceptional cases, PS exposure is generally assumed to mark a point in apoptosis beyond which cells cannot recover, and therefore to be irreversible.

Ablation of normal lipid asymmetry may happen by several means, none of which are well understood, and mechanisms may vary dependent on the stimulus. Rapid loss of lipid asymmetry, also known as lipid ‘scrambling’, can be stimulated experimentally by treatment with calcium ionophore. Whilst it was originally thought that the process was dependent on the activity of a phospholipid scramblase mediating non-specific bi-directional transport of phospholipids, knock-out mice, in which the gene encoding phospholipid Scramblase 1 (PLSCR 1) was deleted, exhibited no defect in PS externalisation (Zhou et al, 2002). More recently it has been suggested that the ATP binding cassette transporter, ABCA1 might act as an outwardly-directed PS translocase (Hamon et al, 2000), though whether this protein directly transports PS or acts upstream in the process is unclear.

Cell surface receptors for ATP can be divided into two classes: metabotropic and ionotropic. The ionotropic class (named “P2X” receptors), comprise seven known members, P2X₁-P2X₇. They are ligand-gated ion channels. They are believed to be multisubunit proteins, with two transmembrane domains per subunit (Buell et al, 1996 Europ. J. Neurosci. 8, 2221).

P2X₇ is the most recently identified member of the P2X family. It is structurally related to the other P2X receptors, but has a significantly longer cytoplasmic C-terminus. Upon activation, the P2X₇ receptor opens a channel through the cell membrane, which channel is permeable to small cations (such as Na⁺ or K⁺). It is not yet clear whether the P2X₇ receptor itself actually forms the channel or acts on a heterologous pore-forming polypeptide.

Expression of the purinergic receptor P2X₇ is largely restricted to haematopoietic cells (Labasi et al, 2002 J. Immunol. 168, 6436-6445). Activation of P2X₇ receptors by ATP (or more potently benzoylbenzoyl ATP (BzATP)) induces reversible “flopping” of phosphatidylserine (PS) to the outer leaflet of the cell membrane and release of IL-1β (MacKenzie et al, 2001 Immunity 5, 825-835). Genetic loss of P2X₇ receptors results in decreased IL-1β secretion and attenuation of inflammatory responses, whilst human polymorphisms in the P2X₇ gene are associated either with susceptibility to, or survival of, chronic lymphocytic leukemia (CLL). A further characteristic of P2X₇ receptor activation is that the lymphocyte plasma membrane becomes permeable to many fluorochromes of up to 300 Da (Ralevic et al, 1998 Pharmacol. Rev. 50, 413-492). This has commonly been interpreted as indicating the conversion of a cation-selective channel into a cation-selective pore (e.g. as described in U.S. Pat. No. 6,509,163).

The efflux of cationic hydrophobic molecules from mammalian cells is commonly mediated by a subset of ATP-binding cassette (ABC) transporters, including the multidrug resistance P-glycoprotein (P-gp; MDR1; ABCB1); members of the multidrug-resistance associated (MRP; ABCC) family of proteins; and the mitoxantrone resistance protein (ABCG2; also called breast cancer resistance protein, BCRP). The primary physiological role of these proteins is cellular defence against toxic molecules. However, these “efflux” proteins have an exceedingly broad range of substrates and also act to eliminate therapeutic drug compounds from target cells, thereby preventing the drug from attaining a therapeutically effective intracellular concentration. This phenomenon is especially observed, for example, in attempts to treat cancers with cytotoxic drugs, and represents a significant clinical obstacle.

### SUMMARY OF THE INVENTION

In its widest terms, the present invention provides a method of regulating the activity of one or more selected membrane proteins, the method comprising the step of contacting or mixing a lipid membrane comprising the selected membrane proteins with a substance which causes a rearrangement of at least part of the components of the membrane, which rearrangement results in an alteration of the activity of the selected proteins.

A “membrane protein” is a molecule which comprises one or more polypeptides and which is inserted within, or in some way attached to, a membrane lipid bilayer. The term “membrane protein” is intended to encompass membrane lipoproteins, glycoproteins and other modified proteins. The lipid membrane is typically a eukaryotic cell membrane, but may also be part of a cell membrane system e.g. a membrane of the endoplasmic reticulum, golgi body, vesicle etc. or may be a synthetic (acellular) membrane, such as a liposome.

Eukaryotic cell membranes typically comprise a complex mix of lipids, glycolipids, phospholipids and the like. The present inventors have found that (a) it is possible to cause a rearrangement of at least part of the components of the membrane and that (b) such rearrangement can alter the activity of membrane proteins on or in the membrane.

The rearrangement of the components of the membrane may, in particular, involve a redistribution between the inner and outer leaflets of the membrane bilayer. More specifically, the rearrangement preferably involves at least one phospholipid, preferably phosphatidylserine (PS). In at least one embodiment, the invention involves contacting a cell membrane with a substance which causes a (preferably reversible) net redistribution of phosphatidylserine from the inner leaflet to the outer leaflet. This redistribution of phosphatidylserine can be prolonged by contacting the cell membrane with a further substance which binds to phosphatidylserine and retains it in the outer leaflet. An example of such a further substance is Annexin V or a PS-specific antibody or fragment thereof.

The membrane protein whose activity is regulated may be any membrane protein which is affected by the rearrangement of lipid (which term, in the present context, also encompasses phospholipid, glycolipid etc). One particular group of proteins which may be regulated in this way includes “efflux proteins” such as P-glycoprotein. The method of the invention may be used either to up- or down-regulate activity of the membrane proteins. The inventors propose that there are three mechanisms whereby loss of membrane asymmetry might affect any given plasma membrane-associated function:
i) changes in direct interaction between lipids and proteins;
ii) changes in indirect effect of plasma membrane on protein-protein interactions; and
iii) interaction of molecules with, and passive diffusion through, the plasma membrane.

As an illustration, it is known for example that membrane lipids play a central role in signal transduction through their interaction with proteins. There are many examples of this, e.g.
a) The membrane lipid phosphatidylinositol (4,5) P₂ is a substrate for phospholipase γ and β, its hydrolysis (following receptor activation) resulting in the production of the key signalling molecules inositol triphosphate and diacylglycerol.
b) The sphingolipid metabolites ceramide, sphingosine and sphingosine 1-phosphate are regulators of cell proliferation and apoptosis. Ceramide, for example is a potent apoptosis mediator, triggering several signalling pathways such as the stress-activated protein kinase cascade. As several antitumour agents induce ceramide production it follows that any alteration of drug/membrane interactions may modulate cytotoxic effects independent of the level of intracellular drug accumulation.
c) Most protein kinase C (PKC) isoforms require PS and calcium for their activation. It follows that a reduction of the concentration of PS in the cytoplasmic leaflet of the plasma membrane is likely to modulate PKC-dependent activities.

In one specific embodiment the invention may be used, for example, to inhibit the activity of a membrane efflux protein.

One particular embodiment of the invention is discussed in greater detail below.

The present inventors have surprisingly discovered that activation or stimulation of the P2X₇ receptor has the effect of increasing the intracellular concentration of exogenously administered substances, such as therapeutic drugs. In many instances, but not necessarily all, this effect has been shown by the inventors to be mediated by inhibiting the action of cell membrane efflux proteins.

Thus in one aspect the invention provides a method of increasing the effective intracellular concentration of a therapeutic molecule within a cell expressing a P2X₇ receptor, the method comprising the step of contacting the cell with the therapeutic molecule and with a substance which stimulates the P2X₇ receptor. In a preferred embodiment the invention provides a method of inhibiting the action of a cell membrane efflux protein, the method comprising the step of contacting a cell expressing a P2X₇ receptor with a substance which causes activation of the P2X₇ receptor.

A number of efflux proteins are known. These are generally ATP-binding cassette (“ABC”) transporter proteins, and include: P-glycoprotein (also known as P-gp, MDR1 or multidrug resistance 1; and ABCB1); members of the multidrug-resistance associated family of proteins (exemplified by MRP and ABCC); and mitoxantrone resistance proteins (ABCG2, also known as breast cancer resistance protein, BRCP).

The method of the invention may be performed in vitro or in vivo. Use of the method in vitro may be advantageous, for example, to facilitate the study of the effect of a particular substance on a cell or tissue, which substance might otherwise be removed from the cell by efflux proteins so as to prevent an effective concentration from being attained. Use of the method in vivo may be especially advantageous to facilitate delivery of therapeutic drugs to intracellular targets.

Substances which stimulate the P2X₇ receptor include P2X₇ agonists such as ATP and ATP analogues, such as benzoylbenzoyl ATP (abbreviated as BzATP). These compounds are stimulatory for the P2X₇ receptor at concentrations which are non-toxic. BzATP is preferred to ATP, as being both more specific for the P2X₇ receptor, and possessing greater receptor-stimulating activity.

Other substances which the inventors predict might stimulate the P2X₇ receptor include antibodies and antibody-like variants (such as Fv, scFv, Fab etc.) with specific binding affinity for the P2X₇ receptor. More especially divalent or multivalent antibodies and the like are preferred. Binding of antibodies or antibody-like molecules to the surface-exposed portion of the P2X₇ receptor is predicted to cause stimulation thereof. The use of such P2X₇-specific stimulants may be advantageous, since they can specifically target P2X₇, whereas more conventional agonists such as ATP will have a wide range of effects in addition to stimulating the P2X₇ receptor. Other modulators of the P2X₇ receptor include NAD (see Seman et al, Immunity, 2003 19, 571-582) and the peptide LL37 (see Elssner et al, J. Immunol. 2004 172, 4987-4994).

Antibodies specific for the P2X₇ receptor can readily be made by the person skilled in the art. Recombinant P2X₇ can, for instance, be readily expressed in a suitable host cell (e.g. as described in U.S. Pat. No. 6,509,163). The protein can be readily purified e.g. by epitope tagging and passage through an affinity chromatography column. A hexahistidine tag is commonly used for this purpose. The purified protein can then be used to generate polyclonal antisera or monoclonal antibodies by conventional techniques (e.g. as described by Sambrook et al, Molecular Cloning: A Laboratory Manual, Cold Spring Harbor Press, Cold Spring Harbor, USA).

As explained above, the inventors have surprisingly found that stimulation of the P2X₇ receptor results in inhibition of various efflux proteins including, in particular, P-gp. Accordingly the method of the first aspect of the invention may be especially useful in facilitating chemotherapeutic treatment, by increasing the effective intracellular concentration of drugs within cells which express the P2X₇ receptor and an efflux protein which is inhibited by P2X₇ receptor stimulation.

Accordingly in a preferred embodiment, the invention provides a method of increasing the effective intracellular concentration of a therapeutic molecule within a cell expressing a P2X₇ receptor and an efflux protein which is inhibited by stimulation of the P2X₇ receptor, the method comprising the steps of contacting the cell with the therapeutic molecule and with a substance which stimulates the P2X₇ receptor. It will be appreciated that, in this context, the therapeutic molecule may be a moiety intended to correct a deficiency or defect in the cell and thereby protect the cell, or may be a cytotoxic drug intended to kill the cell (e.g. because the cell is malignantly transformed), so the term “therapeutic” should be construed accordingly. Equally, the “therapeutic” molecule may not itself be the active agent but may be an inactive precursor which is converted into the active agent, for example, inside the cell. Again, the term “therapeutic” must be construed accordingly.

It will be appreciated that the P2X₇ receptor stimulating substance may be co-administered with the therapeutic molecule or administered separately. The time interval between administration of the two agents which is permissible will depend, at least in part, on the pharmacokinetic profile (e.g. half-life in vivo) of the therapeutic molecule.

The P2X₇ receptor is expressed primarily on haemopoietic cells, such as lymphocytes, macrophages, and the like. Thus, the invention will be especially useful in facilitating treatment of disease in these cells. Examples of particular interest are the facilitation of treatment of malignancies such as leukaemias, autoimmune disorders and allergies. Numerous drugs have been used to treat diseases of this sort, and which are known to be, or suspected of being, exported from the cell by efflux proteins such as P-gp.

Examples of such drugs include the following: cimetidine, colchicine, cyclosporin, dexamethasone, mitomycin, terfernadine, vinblastine, simvastatin and vincristine. This is a non-exhaustive list, and there are many other drugs which may benefit from the present invention by administration generally simultaneously with a P2X₇ receptor stimulating substance.

For present purposes, a drug and a P2X₇ receptor stimulating substance may be considered to be administered “generally simultaneously” if there is a time point in which both substances are present in the subject at an effective concentration.

The drug may be administered by any conventional route e.g. intravenous, intramuscular or subcutaneous injection; or by nasal, oral or rectal administration. The P2X₇ receptor stimulating substance may likewise be administered by any conventional route as aforementioned, although generally an injectable route will be preferred.

A suitable dose of P2X₇ receptor stimulating substance will depend on the activity of the substance and the route of administration. For example, BzATP has been injected into the hindpaw of rats at a concentration of 100 nmol/50 μl and found to be non-toxic (Wismer et al, 2003 Brain Research 965, 187-193). A suitable dose in a human subject would probably be such as to achieve a plasma concentration of 10 nmol-10 mM, preferably in the range 100 nmol-1 mM. An amount suitable to achieve this concentration can readily be determined by routine trial and error experimentation.

Use of the method of the invention to inhibit efflux proteins enables a higher effective intracellular concentration of drug to be attained within P2X₇-expressing cells for a given dose of drug. Alternatively, the dose of drug can be reduced and the effective intracellular concentration can be maintained—this is likely to reduce the severity and/or incidence of adverse reactions to the drug.

In another aspect the invention provides a pharmaceutical composition for administration to a mammalian subject, the composition comprising: a therapeutic drug; a P2X₇ receptor—stimulating substance; and a physiologically acceptable carrier, diluent or excipient. Numerous suitable carriers, diluents or excipients are known to those skilled in the art, and include for example sterile water, saline, phosphate-buffered saline, calcium carbonate, silica, starch, gelatin and the like.

In a further aspect the invention provides a method of making a pharmaceutical composition, comprising the step of combining in admixture a therapeutic drug, a P2X₇ receptor-modulating substance, and a physiologically acceptable carrier, diluent or excipient.

In yet another aspect the invention provides for the use of a P2X₇ receptor-modulating substance in the preparation of a pharmaceutical composition to inhibit an efflux protein in P2X₇ receptor expressing cells.

The inventors have also found that there is an inverse correlation between the level of expression of CD45 (a tyrosine phosphatase) and the amount of PS translocation. Accordingly the inventors predict that inhibiting the activity and/or level of expression of CD45 in cells should decrease the threshold concentration of P2X₇ agonist substance required to produce observable effects (in terms of PS translocation and/or efflux protein inhibition). Accordingly, in the various method/composition aspects of the present invention, it may be advantageous to include a substance which inhibits the activity and/or expression of CD45. A number of inhibitors of CD45 are known (see, for example, Chen & Seto 2004 Bioorg. Med. Chem. 12, 3289-98; Wang et al 2004 Biochem. 43, 4294-4303; and McCain et al, 2004 J. Biol. Chem. 279, 14713-14725).

It is conceivable that rearrangement of lipid within the cell membrane of lymphocytes can be achieved by means which do not require stimulation of the P2X₇ receptor. For example, binding of a ligand to the T cell receptor (TCR) may trigger lipid rearrangement. The ligand may be for example an antibody or antibody-like molecule with specific binding activity for the TCR. Alternatively antigen-stimulation may possibly have a similar effect—this would be extremely useful for causing lipid rearrangement in the cell membrane of B cells, since B cells express the P2X₇ receptor at only a relatively low level, so P2X₇ receptor stimulation is not as effective in B cells as in T cells.

The invention will now be further described by way of illustrative example and with reference to the accompanying drawings, in which:

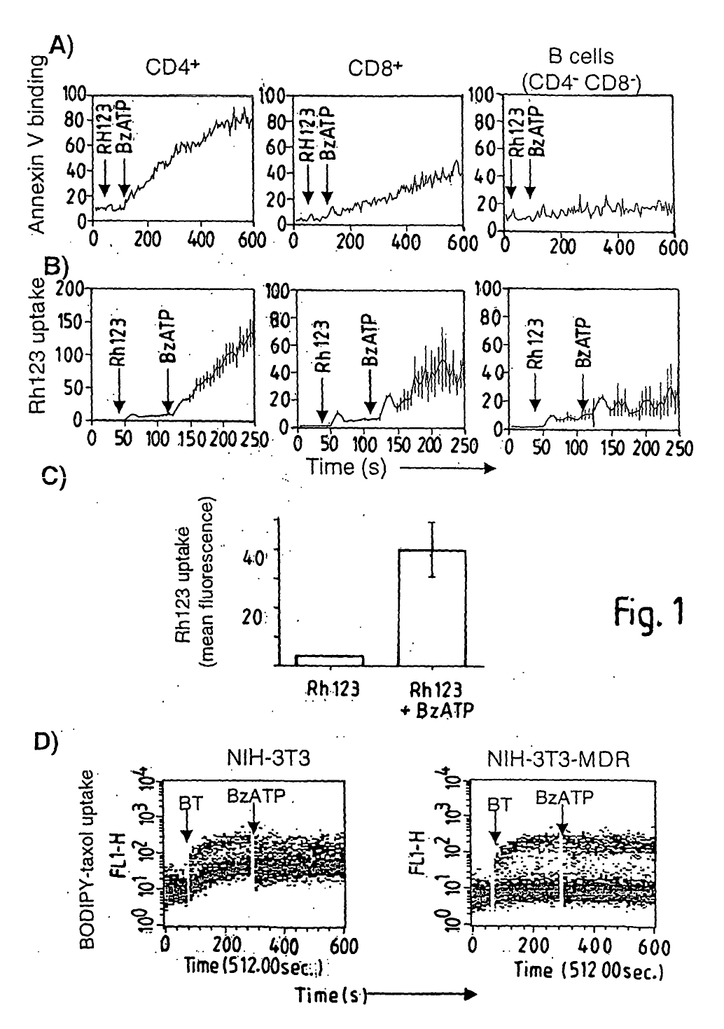

FIG. 1A shows a graph of annexin V binding (as judged by mean fluorescence) against time for CD4⁺, CD8⁺ and CD4⁻ CD8⁻ (primarily B) cells;

FIG. 1B shows inhibition of P-gp activity (as measured by increased Rh123-mediated fluorescence) against time for the same three different cell populations;

FIG. 1C is a bar chart comparing Rh123 uptake in cells with or without treatment with BzATP;

FIG. 1D shows graphs of BODIPY-taxol uptake (as judged by fluorescence) against time for NIH-3T3 cells or NIH-3T3-MDR cells, which do not express significant levels of the P2X₇ receptor;

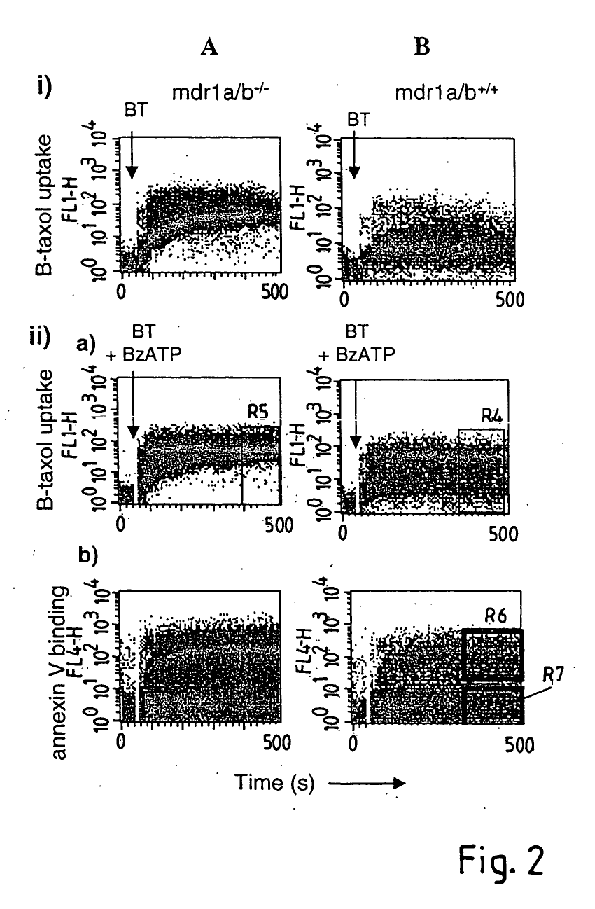

FIGS. 2(i) and 2(ii) a/b are graphs showing binding or uptake, as appropriate, (against time) of particular fluorochrome-labelled substances by various cell populations under different experimental conditions;

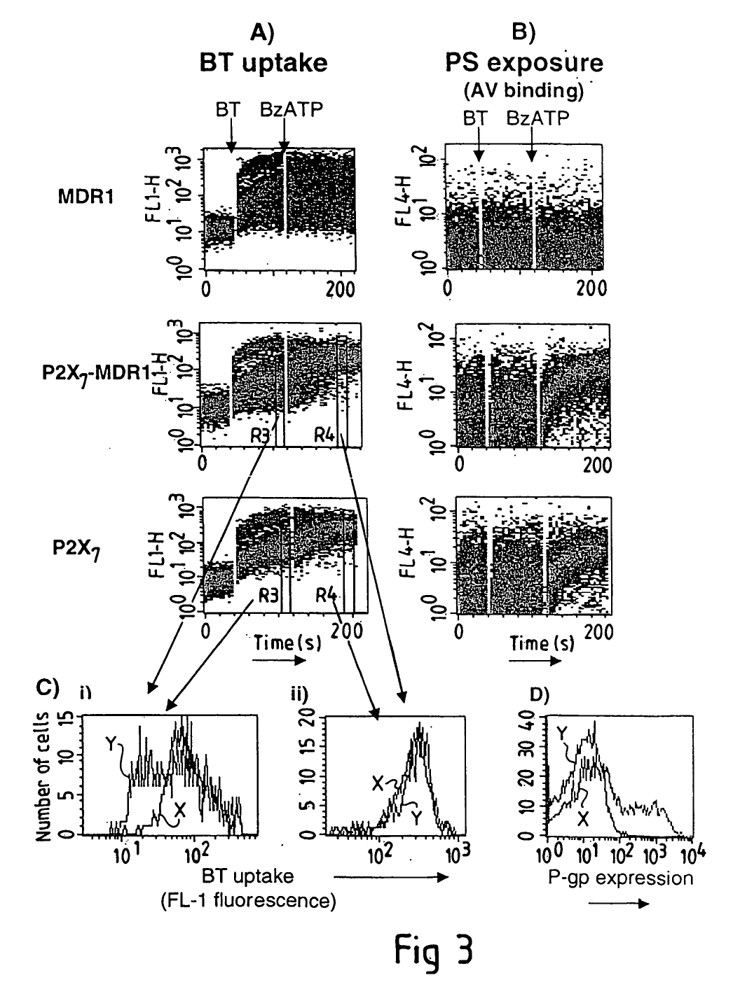

FIGS. 3(i)-(iii) column A are graphs showing BODIPY-taxol uptake against time for three different cell populations. FIGS. 3(i)-(iii) column B show labelled annexin V binding (a measure of PS translocation) against time for the same cell populations as in column A;

FIGS. 3C(i) and (ii) are an alternative representation of some of the data shown in FIGS. 3(ii)A and 3(iii)A;

FIG. 3D is a graph of number of cells against P-gp expression;

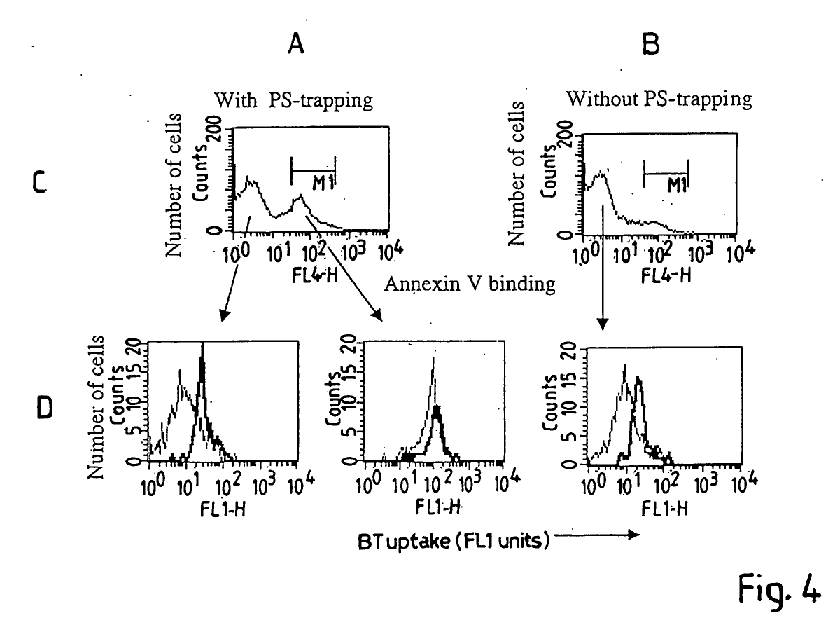

FIGS. 4A-D are histograms showing uptake/binding of various fluorochromes by cell populations under different experimental conditions;

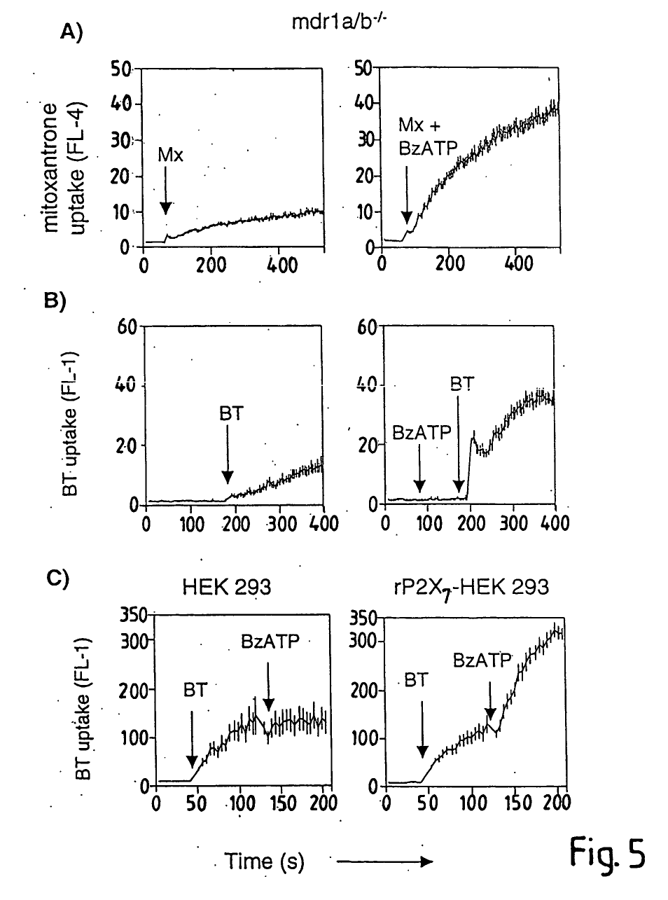

FIGS. 5A-C are graphs showing uptake (against time in seconds) of particular fluorochromes by various cell populations under different experimental conditions; and

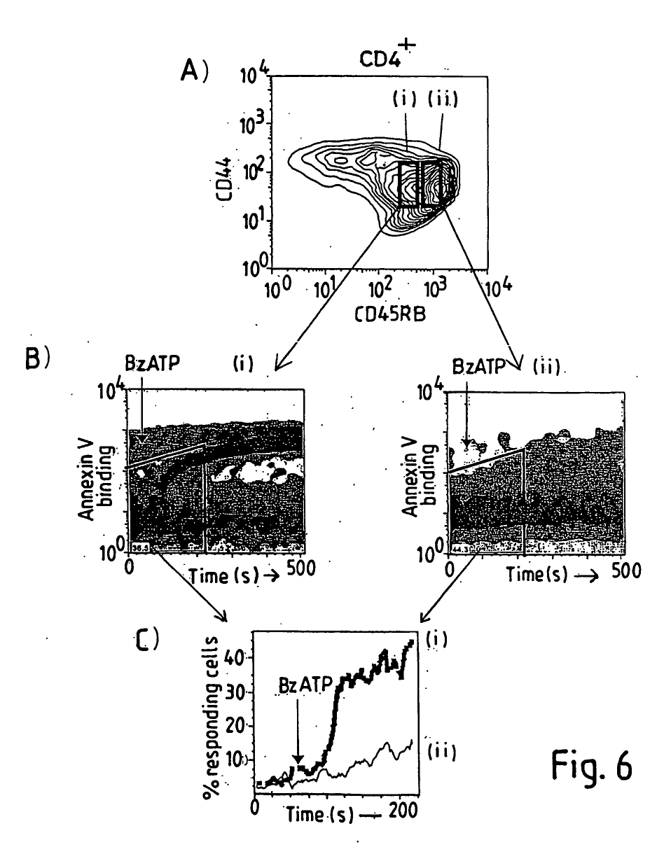

FIG. 6A-C are graphs showing the relationship between the rate of P2X₇-dependent PS translocation and the level of cell surface CD45RB on CD4⁺ T cells.

### EXAMPLES

1. Materials and Methods

Mice

Mdr1a/mdr1b double knockout mutations in mice (Schinkel et al, 1997 Proc. Natl. Acad. Sci. USA 94, 4028-4033) were backcrossed for at least seven generations onto the FVB background at Taconic Farms (Germantown, USA). Mice were between 6 and 14 weeks of age. When experiments utilised FVB.mdr1a/b^{-/-} mice, age-matched FVB mice were used.

Cells

NIH 3T3 cells stably transfected with the human MDR1 gene (Ueda et al, 1987 Proc. Natl. Acad. Sci. USA 84, 3004-3008) were a gift from Michael Gottesman (NIH, Bethesda) and were maintained in 1 μg/ml colchicine in Dulbecco's modified Eagle's medium (DMEM—Sigma). HEK 293 cells stably transfected with rP2X₇ have been described elsewhere (Wilson et al, 2002 J. Biol. Chem. 277, 34017-34023). Transient transfection of HEK293 cells was performed using polyethyleneimine (Sigma), as described previously (Dixon et al, 2000 Hum. Mol. Genet. 9, 1209-1217). pMDR1-wt, which encodes wild-type P-gp, has also been described previously (Blott et al, 1999 EMBO J. 18, 6800-6808). Expression of the MDR1 gene product, P-gp, on transfected cells was determined by labelling of cells with UIC2^{PE} in the presence of cyclosporine A (Sigma), as described by Mechetner et al, (1997 Proc. Natl. Acad. Sci. 94, 12908-12913).

Fluorochrome Efflux Assays

Murine mesenteric lymph nodes (10⁷/ml) were disaggregated in DMEM. To discriminate between different lymphocyte subsets, cells were stained with CD4^{APC}, CD4^{CYCHROME}, CD4^{PE}, CD4^{FITC}, CD8^{APC}, CD8^{PE}, CD8^{PERCP}, CD8^{FITC}, B220^{APC} (Becton Dickinson, Cailf.) antibodies as indicated. The fluorophore on antibodies used to identify CD4⁺ and CD8⁺T cells was varied in some experiments to avoid overlap between emission spectra of the antibody-conjugated fluorochromes and fluorescent transport substrates. Cells were washed with DMEM and analysed by flow cytometry (CellQuest software—Becton Dickinson). Live cells were gated by eye on the basis of forward scatter and side scatter. Lymphocytes lacking CD4 and CD8 were defined as B cells.

To measure drug uptake the following fluorochromes were used (from Molecular Probes, Leiden, Netherlands, unless stated): 0.2 μM BODIPY-taxol (BT), 0.25 μM Rh123, 0.2 μM mitoxantrone (Sigma). PS exposure was monitored simultaneously in the same population of cells by increased binding of annexin V as follows. Cells were equilibrated with annexin V^{FITC}, annexin V^{PE}, or annexin V^{CY5} (AV—Becton Dickinson, Cailf.) together with propidium iodide (PI) for four minutes, and analysed by flow cytometry on a FACScalibur machine using CellQuest software (Becton Dickinson, Cailf.). Baseline fluorescence was established for approximately one minute prior to addition of 150 μM (unless otherwise stated) 2′-3′-O-(4-benzoylbenzoyl)-adenosine 5′-triphosphate (BzATP, Sigma). Cells were monitored for BT uptake or PS exposure continuously in real time for up to 15 minutes, as indicated. Continuous line graphs of changes in annexin V binding and fluorochrome uptake were plotted using FCSPress software (FCSPress.com, Cambridge, UK).

Murine T lymphocytes express both the P2X₇ receptor (Chused et al, 1996 J. Immunol. 157, 1371-1380) and P-gp (Bommhardt et al, 1994 Eur. J. Immunol. 24, 2974-2981). To assess whether P2X₇-stimulation results in PS translocation by murine T cells, and the potential effect of phosphatidylserine (PS) translocation on P-gp function, the inventors monitored both PS translocation and the uptake of the P-gp substrate Rh¹²³, simultaneously in the same cell population, using real-time flow cytometry. The results of these experiments (illustrated in FIGS. 1A-D) showed that activation of the P2X₇ receptor simultaneously stimulates PS translocation within the cell membrane and inhibits P-gp actvity.

Lymphocytes were labelled with anti-CD4^{CYCHROME} and anti-CD8^{APC} antibodies to discriminate between CD4⁺T cells, CD8⁺T cells and B cells (CD4⁻ CD8⁻). The lymphocytes were incubated with annexin V^{PE}, and then 0.25 μM Rh123 and 150 μM BzATP were added sequentially (as indicated in the Figures by arrows).

FIG. 1A shows increases in extracellular PS exposure in each cell population, as indicated by increased biding of annexin V^{PE}. It is clear from FIG. 1A that BzATP stimulated PS translocation with the order of responsiveness CD4⁺ cells>CD8⁺ T cells>>B cells (FIG. 1A), consistent with the known pattern of expression of P2X₇ receptors.

FIG. 1B shows P-gp activity, measured simultaneously in the same population of cells, as indicated by uptake of Rh123 (increase in mean (±S.E.) fluorescence). Note that an increase in Rh123 uptake (fluorescence increase) reflects an inhibition of P-gp activity. Again, the order of responsiveness was CD4⁺>CD8⁺>>B cells.

FIG. 1C shows the results obtained when lymphocytes were labelled with anti-CD4^{PE}, incubated with annexin V^{CY5} and then 0.2 μM Rh123 added in the presence (n=5) or absence (n=4) of 150 μM BzATP. (The histogram shows the uptake of Rh123 (increase in mean (±S.D.)) at 3 minutes.)

FIG. 1D shows that BzATP does not affect P-gp activity in NIH3T3 cells which lack the P2X₇ receptor. BODIPY-taxol (BT) and 175 μM BzATP were added to NIH3T3-MDR and parental NIH3T3 cells at times indicated by arrows. BT uptake was monitored by continuous FACS analysis, as an increase in fluorescence as a function of time. As expected, BT did not accumulate in NIH3T3-MDR cells due to P-gp activity. In contrast to lymphocytes (FIGS. 1A and B), BzATP did not inhibit P-gp activity and thus did not increase BT accumulation. (Note: in these and other experiments utilising cultured cells, a small proportion of cells rapidly accumulated very high levels of BODIPY-taxol and were unresponsive to BzATP; it was unclear what such cells (or cell debris) represented, and they were not analysed further).

In a further experiment, lymphocytes from mdr1a/b-deficient or parental mice (mdr1a/b^{+/+}) were labelled with anti-CD8^{PE} and anti-CD8^{PERCP} antibodies, respectively (to discriminate between CD8⁺ cells from each mouse during subsequent analysis). The cells from each mouse were mixed, and BODIPY-taxol and annexin V^{CY5} added to enable simultaneous monitoring by flow cytometry of PS translocation and P-gp activity in real time in the same population of cells. Reduced BT uptake is indicative of enhanced P-gp activity; increased annexin^{CY5} binding indicates increased PS translocation from the inner to outer leaflet of the cell membrane. The results are shown in FIGS. 2(i) and (ii).

FIGS. 2(i) and 2(ii) (a/b) are graphs showing BT uptake (as measured by fluorescence) against time for mdr1a/b-deficient mice (column A) or parental mice (column B). FIG. 2(i) shows the BT uptake in untreated cells. It is apparent that BT uptake in the cells from the parental mouse is much lower than that in cells from the mdr-deficient mice, as expected, indicating that BT uptake is limited by P-gp as reported previously (Binaschi et al, 1995 Int. J. Cancer 62, 84-89).

FIGS. 2(ii) (a) and (b) show, respectively, BT uptake and PS translocation following stimulation by addition of BzATP. BT uptake following BzATP treatment rapidly reached equivalence in the mdr-deficient and wild type cells. (Note that, at the concentration of BzATP employed, a minority of cells failed to translocate PS: in these cells BT uptake remained greater in the mdr-deficient cells, thus strengthening the correlation between PS translocation and inhibition of P-gp activity.)

To confirm that BzATP was acting through P2X₇ receptors, the effect of P2X₇-stimulation on P-gp activity was studied on HEK 293 cells stably transfected with the P2X₇ gene. As HEK cells do not express P-gp, MDR1 cDNA was transiently transfected into the cells. The cells were equilibrated with annexin V^{CY} and propidium iodide. The results are shown in FIGS. 3(i)-(iii) which are graphs of fluorescence against time. Columns A and B show the results for BT uptake and PS translocation respectively. Only the responses of live cells (i.e. those excluding propidium iodide) were analysed.

BzATP induced PS translocation and concomitantly increased BODIPY-taxol uptake (i.e. inhibited P-gp) in cells expressing both P-gp and P2X₇ receptor (FIG. 3(ii)) but neither induced PS translocation nor increased BODIPY-taxol uptake in cells expressing P-gp but lacking P2X₇ receptors (FIG. 3(i)). Thus, only in P2X₇-expressing cells did BzATP inhibit P-gp activity, confirming that BzATP acts through the P2X₇ receptor.

As observed in FIG. 1 a small proportion of cultured cells (or cell debris) rapidly accumulated very high levels of BT and were unresponsive to BzATP treatment. These were excluded from the histogram analysis presented in FIGS. 3C and D.

FIG. 3C is an alternative plot of data in FIGS. 3(ii)A and 3(iii)A, and shows a comparison of steady-state BODIPY-taxol uptake by mock transfected P2X₇ cells (“X”) and P2X₇-MDR cells (“Y”). (i) before BzATP stimulation (indicated by gates R3), or (ii) following BZATP stimulation (indicated by gates R4).

FIG. 3D shows P-gp expression by mock-transfected HEK 293 -P2X₇ cells (“X”), and HEK 293-P2X₇ cells transiently transfected with the MDR1 gene (“Y”) as measured by binding of fluorescently-conjugated, P-gp-specific antibody (UIC2^{PE}). An equivalent proportion (⁻35%) of P2X₇-MDR1 cells expressed P-gp (3D) as were able to exclude BODIPY-taxol (FIG. 3Ci).

In this example, the inventors compared the uptake of BODIPY-taxol by parental and mdr1a/b-deficient lymphocytes, briefly stimulated with BzATP either in the continuous presence of annexin V to ‘trap’ PS in the outer leaflet, or with annexin V added only shortly before acquisition. The results are shown in FIGS. 4A-D. The experiments were performed as described below.

Lymphocytes from mdr1a/b-deficient or parental mice (mdr1a/b^{+/+}) were labelled with anti-CD8^{PE} and anti-CD8^{CY} antibodies, respectively (to discriminate between CD8⁺ cells from each mouse during subsequent analysis). The cells from each mouse were mixed, stimulated with BzATP for 45 seconds, washed, and incubated with annexin V either for 30 minutes to ‘trap’ PS in the outer leaflet (column A), or for only the final 4 of 30 minutes (column B) to allow detection of PS prior to acquisition. BODIPY-taxol uptake was then measured by real-time flow cytometry. Reduced BT uptake indicates enhanced P-gp activity; increased annexin^{CY5} binding indicates increased PS translocation from the inner to outer leaflet of the membrane. Row C shows histograms comparing annexin V binding at steady state (after 180-220 seconds incubation) and Row D shows BODIPY-taxol accumulated by mdr1a/b deficient (thick line) and mdr1a/b^{+/+} (thin line) lymphocytes in the same population of cells at the same timepoint gated on the basis of either high or low levels of external PS. As PS exposure in samples stained with annexin V for four minutes immediately following stimulation (not shown) was equivalent to that in cells incubated for 30 minutes, PS trapping by annexin V appeared to be effectively complete.

The data demonstrate that annexin V treatment prevents the restoration of P-gp activity.

P-glycoprotein is only one of a family of transporters that can provide protection from drugs (Allen et al, 2000 Cancer Res. 60, 5761-5766). The inventors therefore examined whether P2X₇-stimulation may also inhibit drug efflux activity by transporters other than P-gp. Mitoxantrone efflux, presumed to be mediated by the breast cancer resistance protein (abcg2), was found in CD4⁺ (and to a lesser extent CD8⁺ T cells). The results are shown in FIGS. 5A-C.

FIG. 5A shows the results obtained when lymphocytes from mdr1a/b-deficient mice were labelled with anti-CD4^{FITC} to gate CD4⁺ T cells. Mitoxantrone (Mx) or Mx plus BzATP, were added as indicted by arrows. Mitoxantrone uptake was measured as an increase in mean (±S.E.) fluorescence (FL-4 channel). FIG. 5B shows results obtained when lymphocytes from mdr1a/b-deficient mice were labelled with anti-CD8^{PERCP}, and BODIPY-taxol or BODIPY-taxol plus 175 μM BZATP added as indicated. BODIPY-taxol uptake was measured as an increase in mean (±S.E.) fluorescence (FL-1 channel). FIG. 5C similarly illustrates results obtained when HEK 293 and P2X₇-HEK 293 cells were equilibrated with propidium iodide. BODIPY-taxol (BT) and 150 μM BzATP were added, as indicated. Only the responses of live cells (i.e. those excluding PI) were subsequently analysed.

The Figures show that BzATP stimulation of P2X₇ induced PS translocation and markedly increased uptake of mitoxantrone (MW 481) in mdr1a/b-deficient lymphocytes, indicating that loss of plasma membrane asymmetry may be associated with inhibition of transporters in addition to P-gp. Though P-gp is the only characterised transporter for BODIPY-taxol, BzATP also increased the rate of uptake of this fluorochrome in mdr1a/b-deficient CD4⁺ and CD8⁺T cells, and in P2X₇ HEK 293, but not in control cells. Loss of membrane asymmetry may therefore also increase drug uptake by routes independent to effects on known multidrug transporters.

P-gp protects normal lymphocytes from toxins, and by pumping drugs from cells it can reduce the efficacy of chemotherapy. Expression of P-gp is most commonly associated with multidrug resistance on cancers, but it also provides potential barriers to the treatment of many other immune-related disorders from transplant rejection to chronic autoimmune disease and AIDS (Kim et al, 1998 J. Clin. Invest. 101, 289-294; Fellay et al, 2002 Lancet 359, 30-36). Concomitant with P2X₇-stimulated PS translocation, lymphocytes markedly increased uptake of the P-gp substrates Rh123 (MW 381) and BODIPY-taxol (MW 1024), but not of the non-P-gp substrate propidium iodide (MW 414). Hence, P2X₇-stimulation results in selective increase in drug uptake consistent with loss of P-gp activity. The inventors' findings suggest the apparent increase in permeability is due (at least in part) to inhibition of efflux pumps. As P2X₇ activation increases ‘flopping’ of PS to the extracellular leaflet of the membrane simultaneously with the inhibition of P-gp activity, a plausible interpretation of the data is that loss of plasma membrane lipid asymmetry inhibits P-gp activity by preventing the structural changes of the P-gp molecule required for drug efflux (Rosenberg et al, 2001 EMBO J. 20, 5615-5625). Alternatively, altered membrane asymmetry may alter the distribution of drugs in the lipid phase such that they cannot access the substrate biding site of P-gp (Higgins & Gottesman 1992 Trends Biochem. Sci. 17, 18-21).

It is reasonable to assume that P2X₇-stimulation results not only in the flopping of PS to the outer leaflet of the plasma membrane, but also a broader lipid redistribution. Were this not the case it is likely that the resulting excess phospholipid content in the outer leaflet would result in rapid membrane disruption. It is also likely that unidirectional lipid translocation would be highly energetically unfavourable. It follows that there is a high probability any process dependent on membrane lipids will be modulated by loss of lipid asymmetry.

The potential of pharmacological inhibitors of P-gp activity to reverse multidrug resistance in cancers has been severely limited by the consequent elevated drug entry into organs such as the brain that are protected by drug transporter activity (Schinkel 2001 Adv. Exp. Med. Biol. 500, 365-372). As P2X₇-stimulation inhibits the activity of P-gp and probably also of other drug transporters, and the receptor is relatively specific to lymphocytes, its agonists may increase therapeutic drug uptake in lymphocytes but not in other tissues. Co-administration of P2X₇ agonists would be expected to potentiate lymphoid-specific accumulation of a wide variety of drugs including, but not limited to, anti-cancer agents, HIV protease inhibitors, and immunosuppressants. This should avoid the undesirable consequences of traditional multidrug inhibitors that lead to widely altered, and potentially cytotoxic, drug uptake.

BzATP-mediated stimulation did not result in an increased uptake of propidium ions (MW 414), (data omitted for brevity), hence the observed effects were not simply due to a non-specific increase in plasma membrane permeability.

In this example, the inventors compared the sensitivity of naïve and activated/memory T cells (defined by relative expression levels of CD45RB and CD44) to P2X₇-stimulation.

Murine lymphocytes were labelled with anti-C4^{APC}, anti-CD44^{CHCHROME} and anti-CD45RB^{PE} antibodies, washed and pre-incubated with annexin V^{FITC} to permit analysis of PS exposure. FIG. 6A shows a contour plot of CD44 and CD45 RB expression on CD4⁺ cells to identify naive (CD44^{lo}CD45TRB^{hi}) and activated/memory (CD44^{hi}CD45RB^{lo}) CD4⁺ T cells and the gating of sub-populations of naive cells with small differences in CD45RB expression (i and ii).

FIG. 6B shows density plots of the rate of PS exposure (as judged by binding of annexin V) in CD4⁺ (i) CD44^{lo}CD45RB^{int} cells and (ii) CD44^{lo} CD45RB^{hi} cells. BzATP was added to the cell suspension shown in FIG. 6A as indicated by the arrows.

FIG. 6C is an alternative representation of the data shown in FIG. 6B and illustrates the frequency of cells responding to P2X₇-stimulation (annexin binding) in cells within the boxes indicated in FIG. 6B.

Strikingly, it was found that the CD4⁺ T cells were not uniformly sensitive. Rather, responsiveness appeared to be inversely related to the cell surface expression of CD45RB, with even small increments in the level of CD45RB being associated with significant decreases in P2X₇-responsiveness (as observed from comparison of FIG. 6B, parts (i) and (ii)). Therefore, the rate of P2X₇-dependent PS translocation was found to be inversely related to the level of cell surface CD45RB on CD4⁺ T cells.

Thus, in the naïve T cell population, the inventors have found an inverse correlation between CD45 expression and PS exposure following P2X₇-stimulation in CD4⁺ CD45RB^{int/hi} cells. Hence, a continuation exists: on cells expressing the lowest levels of CD45RB, PS is constitutively exposed, whereas increasing amounts of CD45RB expression correlates with an elevated threshold for P2X₇-stimulated PS translocation. Though the lack of antibodies to the extracellular domain of murine P2X₇ precludes direct comparison of CD45 and P2X₇ expression levels, the tight inverse correlation between CD45RB expression and PIX₇ activity was observed both in wild type mice and gene targeted and transgenic mice expressing varying levels of CD45 isoforms. As other data implicate receptor protein tyrosine phosphatase B(RPTP-B) in feedback control of P2X₇-transfected heterologous (HER) cells (Kim et al, 2001), the simplest hyopthesis is that in its native environment P2X₇ is negatively regulated by the tyrosine phosphatase CD45, in addition to, or in place of, RPTP-B.

1. A method of increasing the effective intracellular concentration of a therapeutic molecule within a cell expressing a P2X₇ receptor, the method comprising the step of contacting the cell with the therapeutic molecule and with a substance which modulates the P2X₇ receptor.

2. A method according to claim 1, wherein the method comprises the step of contacting the cell with the therapeutic molecule and with a substance which stimulates the P2X₇ receptor.

3. A method according to claim 1 or 2, wherein the therapeutic molecule is a cytotoxic drug and it is desired to kill the cell.

4. A method according to claim 1, wherein the P2X₇ receptor modulating substance comprises ATP, an analogue of ATP, or an immunoglobulin or immunoglobulin-like variant which possesses specific binding activity for the P2X₇ receptor.

5. A method according to, claim 1 wherein the cell expresses an efflux protein which is inhibited by stimulation of the P2X₇ receptor.

6. A method according to claim 5, wherein the efflux protein which is inhibited is one or more selected from the group consisting of: P-glycoprotein; mitoxantrone resistance protein; and a member of the multidrug-resistance associated family of proteins.

7. A method according to claim 1, wherein the therapeutic molecule and the P2X₇ receptor modulating substance are co-administered.

8. A method according to claim 1, wherein the cell is contacted with a substance which inhibits the activity and/or expression of CD45.

9. An in vitro method in accordance with claim 1.

10. (canceled)

11. (canceled)

12. (canceled)

13. (canceled)

14. A pharmaceutical composition for administration to a mammalian subject, the composition comprising: a therapeutic drug; a P2X₇ receptor modulating substance; and a physiologically acceptable carrier, diluent or excipient.

15. A pharmaceutical composition in accordance with claim 14, the composition comprising: a therapeutic drug; a P2X₇ receptor stimulating substance; and a physiologically acceptable carrier, diluent or excipient.

16. A pharmaceutical composition according to claim 14 or 15, further comprising a substance which inhibits the activity and/or expression of CD45.

17. A method of making a pharmaceutical composition comprising the step of combining in a mixture a therapeutic drug, a P2X₇ receptor modulating substance, and a physiologically acceptable carrier, diluent or excipient.

18. A method of making a pharmaceutical composition in accordance with claim 17, the method comprising the step of combining in a mixture a therapeutic drug, a P2X₇ receptor stimulating substance, and a physiologically acceptable carrier diluent or excipient.

19. A method according to claim 17 or 18, including a substance which inhibits the activity and/or expression of CD45.

20. A method of inhibiting the action of a cell membrane efflux protein, the method comprising the step of contacting a cell expressing a P2X₇ receptor with a substance which causes activation of the P2X₇ receptor.

21. An in vitro method of inhibiting the action of a cell membrane efflux protein in accordance with claim 20.

22. A method of rearranging at least part of the lipid, phospholipid or glycolipid component of a cell membrane which comprises contacting said cell membrane with a P2X₇ receptor modulating substance.
